(19) United States
(12) Patent Application Publication
(10) Pub. No.: US 2026/0306075 A1
(43) Pub. Date: Oct. 8, 2026

(54) ASPIRATION ACCESSORIES FOR BIOPSY DEVICES AND METHODS OF PERFORMING AN ASPIRATION BIOPSY

(71) Applicant: BARD PERIPHERAL VASCULAR, INC., Franklin Lakes, NJ (US)

(72) Inventors: Jeffrey Yue, Shanghai (CN); Yuhao Zhang, Shanghai (CN)

(73) Assignee: BARD PERIPHERAL VASCULAR, INC., Franklin Lakes, NJ (US)

(21) Appl. No.: 19/482,325

(22) PCT Filed: May 8, 2023
(86) PCT No.: PCT/US2023/021344

§ 371 Date: Nov. 7, 2025

(51) Int. Cl. A61B 10/00

(52) U.S. Cl. CPC A61B 10/0045

(57) ABSTRACT

An aspiration accessory includes a syringe barrel having a first end having a first end surface and a second end. The aspiration accessory further includes a plunger slidably engaged within the syringe barrel between an initial position and a suction position. The plunger includes a first end and a second end, and a plurality of flanges are positioned between the first end and the second end. The plurality of flanges are biased radially outward and the syringe barrel compresses the plurality of flanges in the initial position. When the plunger is moved to the suction position, a biasing force on the plurality of flanges causes the plurality of flanges to flex radially outward and engage the first end surface of the syringe barrel, such that the plunger locks in the suction position.

### TECHNICAL FIELD

The present disclosure relates to biopsy devices, and, more particularly, to aspiration accessories for biopsy devices and methods of operating the same.

### BACKGROUND

Fine needle aspiration (“FNA”) biopsy procedures are commonly conducted to collect specimens from a target area in order to determine the presence of cancerous tissue and/or cells. To perform an FNA biopsy, a thin, hollow needle is inserted through the skin and into the target area, and negative pressure in a syringe coupled to the needle causes a small sample of cells and fluid from the target area to be drawn into the syringe. Due to the minimally invasive nature of FNA biopsies, these procedures are amongst the most common testing procedures conducted to identify cancerous tissue and/or cells. However, common FNA biopsy procedures have a number of shortcomings. For example, FNA procedures may only obtain minimal quantities of specimen material when the needle is inserted into the target area. As a result, procedures often require the needle to be inserted into the target area from multiple locations needle to obtain the desired volume of tissue from the target area. Certain FNA procedures (e.g., dealing with dense, hard, and/or calcified nodules) may also call for the needle to be rotated during sampling, which may result increase the complexity of the procedure. Furthermore, many FNA biopsy accessories require particularly designed reusable and/or disposable handles for generating suction within the needle during the biopsy procedure.

### SUMMARY

An object of the present disclosure is to provide an aspiration accessory for a biopsy device that can acquire a desired quantity of tissue via a single insertion into a target area while being easily maneuverable within the target area.

In one aspect, an aspiration accessory is disclosed. The aspiration accessory includes a syringe barrel having a first end having a first end surface and a second end. The aspiration accessory further includes a plunger slidably engaged within the syringe barrel between an initial position and a suction position. The plunger includes a distal end and a proximal end, and a plurality of flanges are positioned between the distal end and the proximal end. The plurality of flanges are biased radially outward and the syringe barrel compresses the plurality of flanges in the initial position. When the plunger is moved to the suction position, a biasing force on the plurality of flanges causes the plurality of flanges to flex radially outward and engage the first end surface of the syringe barrel, such that the plunger locks in the suction position.

In another aspect, a biopsy device is disclosed. The biopsy device may include an aspiration accessory having a syringe barrel having a first end and a second end, where the first end includes a first end surface. The aspiration accessory may further include a plunger being retractable between an initial position and a suction position, where the plunger includes a distal end and a proximal end, and a plurality of flanges positioned between the distal end and the proximal end. The plurality of flanges of the aspiration accessory may be biased radially outward, and the syringe barrel may compress the plurality of flanges in the initial position. The biopsy device may further include a biopsy needle coupled to the second end of the syringe barrel. When the plunger is moved to the suction position, a biasing force on the plurality of flanges causes the plurality of flanges to flex radially outward and engage the first end surface of the syringe barrel, such that the plunger locks in the suction position.

In yet another aspect, a method of performing an aspiration biopsy is disclosed. The method may involve inserting a needle into an aspiration accessory, where the aspiration accessory includes a syringe barrel having a first end surface and a plunger having a plurality of flanges. The plurality of flanges may be biased radially outward, such that the syringe barrel compresses the plurality of flanges in an initial position. The method may further involve positioning the plunger in the initial position, such that the plunger is contained within the syringe barrel. The method may then involve inserting the needle into a target site and retracting the plunger from an initial position to a suction position, such that a biasing force on the plurality of flanges causes the plurality of flanges to flex radially outward and engage the first end surface of the syringe barrel to lock the plunger in the suction position. The method may then involve drawing fluid from the target site through the needle and into the syringe barrel.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 depicts a front-side view of an illustrative aspiration accessory in an initial position, according to one or more embodiments shown and described herein;

FIG. 2 depicts a front-side view of the aspiration accessory of FIG. 1 in a suction position, according to one or more embodiments shown and described herein;

FIG. 3 depicts a front-side view of a plunger of the aspiration accessory of FIG. 1, according to one or more embodiments shown and described herein;

FIG. 4 depicts a perspective view of the aspiration accessory of FIG. 2, according to one or more embodiments shown and described herein; and

FIG. 5 depicts an illustrative flow diagram of a method of performing an aspiration biopsy, according to one or more embodiments shown and described herein.

Reference characters indicate corresponding parts throughout the several views. The exemplifications set out herein illustrate at least one embodiment of the present disclosure, and such exemplifications are not to be construed as limiting the scope of the present disclosure in any manner.

### DETAILED DESCRIPTION

Embodiments disclosed herein relate to aspiration accessories for biopsy devices and methods of performing aspiration biopsies. The disclosed aspiration accessories may include a syringe barrel having a first end and second end, and a plunger having a plurality of flanges disposed within the syringe barrel. The plunger may be translated from an initial position, in which the plurality of flanges are compressed within the syringe barrel, to a suction position, in which the plurality of flanges are biased radially outward to engage the first end of the syringe barrel and lock the plunger in the suction position. By locking the plunger in the suction position using the plurality of flanges, a user may be able to manually control a volume of fluid (e.g., tissue) drawn into the syringe barrel during a biopsy procedure.

As noted herein, embodiments of the present disclosure may be specifically advantageous for acquiring a desired amount of tissue from a target site. The engagement between the plunger and the syringe barrel may allow a suction force to be applied to the target site, such that a user may be able to collect a desired volume of tissue from the target site without creating multiple incisions in the target site. Furthermore, the design of the plunger may allow for easy manual maneuvering of the aspiration accessory during a biopsy procedure. For example, a user may control the plunger using only a single finger (e.g., thumb), while securing the syringe barrel with one or more other fingers, thereby allowing for single-handed use. In these embodiments, the one or more other fingers may be used to rotate the aspiration accessory during the procedure, which may be particularly desirable during procedures in which tissue is being retrieved from a dense, hard, and/or calcified target site.

Embodiments of aspiration accessories, biopsy devices, and methods of performing aspiration biopsies will now be described in more detail herein with reference to the drawings, and where like numbers refer to like structures.

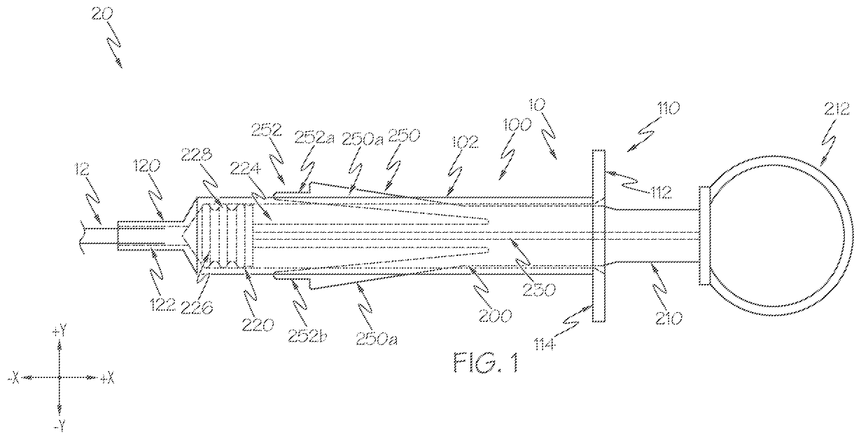

Referring now to FIG. 1, an aspiration accessory 10 is depicted. The aspiration accessory may include a syringe barrel 100 having a first end 110 and a second end 120 opposite the first end 110. In these embodiments, the syringe barrel 100 may include a body 102, such as a hollow body, which may extend between the first end 110 and the second end 120 of the syringe barrel 100.

The first end 110 of the syringe barrel 100 may include a first end surface 112 and a second end surface 114. In these embodiments, the first end surface 112 and the second end surface 114 may extend outwardly (e.g., in the +/-y direction as illustrated in the coordinate axes of FIG. 1) away from the body 102 of the syringe barrel 100. Furthermore, the first end surface 112 may be adjacent an external environment of the aspiration accessory 10, while the second end surface 114 may be adjacent the syringe barrel 100.

Referring still to FIG. 1, the second end 120 of the syringe barrel 100 may include an aperture 122. In these embodiments, a needle 12, such as a fine needle, may be coupled to the aperture 122 to form a biopsy device 20. The needle 12 may be coupled to the aperture 122 such that the needle 12 and the body 102 of the syringe barrel 100 are in fluid communication. For example, the needle 12 and the aperture 122 may be fluidly coupled using any suitable connection (e.g., via threaded connection, adhesive, welding, brazing, etc.). Once the needle 12 is coupled to the aperture 122, fluid from a target site may flow through the needle 12 and into the body 102 of the syringe barrel 100 during a biopsy procedure.

Referring still to FIG. 1, the aspiration accessory 10 may further include a plunger 200. The plunger 200 may be slidably engaged within the body 102 of the syringe barrel 100 such that the plunger 200 may slide in a longitudinal direction (e.g., along the x-axis as depicted in the coordinate axes of FIG. 1) between an initial position and a suction position, as will be described in additional detail herein.

The plunger 200 may include a distal end 210 and a proximal end 220, and a central portion 230 may extend between the distal end 210 and the proximal end 220 of the plunger 200. The plunger 200 may further include a plurality of flanges 250, which may be positioned between the distal end 210 and the proximal end 220 of the plunger 200. For example, in some embodiments, the plurality of flanges 250 may be positioned on the central portion 230 of the plunger.

As further depicted in FIG. 1, the distal end 210 of the plunger 200 may include a sliding mechanism 212. The sliding mechanism 212 may be a ring, such as a finger ring, or any similar mechanism, which allows for manual movement of the plunger 200 in the longitudinal direction. In embodiments in which the sliding mechanism 212 includes a finger ring, a user may insert a finger, such as a thumb, into the finger ring in order to alternate the plunger 200 between the initial position and the suction position, as will be described in additional detail herein.

The proximal end 220 of the plunger 200 may include a stopper fitting 224. In these embodiments, the stopper fitting 224 may be a projection that extends from the central portion 230 of the plunger 200, and may receive a stopper 226. As depicted in FIG. 1, the stopper 226 may be coupled to the stopper fitting 224 such that a sealed engagement with the body 102 of the syringe barrel 100 is achieved. For example, the stopper 226 may be sized such that, when the stopper 226 is affixed to the stopper fitting 224, an outer perimeter of the stopper 26 contacts the body 106 of the syringe barrel 100, such that a seal is formed between the stopper 226 and the body 102 of the syringe barrel 100.

More specifically, the seal formed between the stopper 226 and the body 102 of the syringe barrel may be a fluid-tight seal, such that fluid which enters the body 102 of the syringe barrel 100 via aperture 122 is unable to pass between the stopper 226 and the body 102 to escape from the first end 110 of the syringe barrel 100. It should be understood that the stopper 226 may be coupled to the stopper fitting 224 via any suitable means (e.g., via threaded connection, adhesive, welding, brazing, etc.).

Referring still to FIG. 1, in some embodiments, the stopper 226 may be formed of plastic, such as polypropylene, elastomers, such as rubber, polytetrafluoroethylene (PTFE), or any similar material capable of forming a seal with the body 102 of the syringe barrel 100. In these embodiments, the stopper 226 may be formed of a flexible plastic or rubber, which may aid in affixing the stopper 226 to the stopper fitting 224 and creating the seal between the stopper 226 and the body 102 of the syringe barrel 100.

Furthermore, in some embodiments, it may be advantageous to form the stopper 226 having a low coefficient of friction (e.g., less than 0.15), such as PTFE, as these materials may allow for the stopper 226 to more easily translate in the longitudinal direction within the syringe barrel 100. It should be further appreciated that by allowing the stopper 226 to more easily translate within the syringe barrel 100, a user may be capable of moving the plunger 200 between the initial position and the suction position, which may result in a greater suction force being generated within the syringe barrel 100.

As further depicted in FIG. 1, the stopper 226 may further include a plurality of ribs 228 that may be spaced along a length of the stopper 226. In these embodiments, the plurality of ribs 228 may formed of the same material as the stopper 226, while in some embodiments, the plurality of ribs 228 may formed of a different material. Similarly, the plurality of ribs 228 may be integrally formed with the stopper 226, such that the plurality of ribs 228 and the stopper 226 are a single, monolithic structure, while in other embodiments, the plurality of ribs 228 may be separately affixed to the stopper 226. In the embodiments described herein, a plurality of seals may be formed between the stopper 226 and the body 102 of the syringe barrel 100 where each of the plurality of ribs 228 contacts the body 102 of the syringe barrel 100. By utilizing the plurality of ribs 228 to form a plurality of seals, the stopper 226 may remain sealed to the body 100 of the syringe barrel 100 in the event one or more of the plurality of seals fails. For example, as the plunger 200 is translated within the body 102 of the syringe barrel 100, friction between the body 102 and the plurality of ribs 228 may cause the plurality of ribs 228 to degrade, such that the seal between the body 102 and at least one of the plurality of ribs 228 is broken. However, so long as the remaining seals are not broken, the stopper 226 will remain sealed to the body 102, such that fluid cannot escape into and/or out of the syringe barrel 100, thereby maintaining a vacuum within the body 102 in some embodiments.

Referring still to FIG. 1, and as previously discussed herein, a plurality of flanges 250 may extend from the central portion 230 of the plunger 200. In these embodiments, the plurality of flanges 250 may be biased radially outward (e.g., in the +/−y direction as depicted in FIG. 1), such that the plurality of flanges 250 extend outwardly from the central portion 230 of the plunger 200. As further depicted in FIG. 1, each of the plurality of flanges 250 may further include a locking mechanism 252, such as a shoulder, which may be used to releasably fix the plunger 200 in the suction position, as will be described in additional detail herein. Although the locking mechanism 252 is depicted as a shoulder, it should be understood that the locking mechanism 252 may take any form suitable to releasably fix the plunger 200 in the suction position.

Referring again to FIG. 1, the aspiration accessory 10 is shown in the initial position. In this embodiment, the plurality of flanges 250 of the plunger 200 may include a first flange 250a and a second flange 250b. As depicted, the first flange 250a and the second flange 250b may be compressed within the body 102 of the syringe barrel 100 when the aspiration accessory 10 is in the initial position. Although the aspiration accessory 10 of FIG. 1 is depicted as having a first flange 250a and a second flange 250b, it should be understood that any number of flanges may be utilized. For example, in some embodiments, the plurality of flanges 250 of the plunger 200 may include any number of flanges, such as a single flange, three flanges, four flanges, or any other desired number of flanges.

Furthermore, in the initial position, the stopper 226 of the plunger 200 may partially extend into the aperture 122 of the syringe barrel 100, such that fluid is prevented from entering the body 102 of the syringe barrel 100. In these embodiments, the stopper 226 may include a tapered end, such that the tapered end of the stopper 226 may obstruct the aperture 122 of the syringe barrel 100.

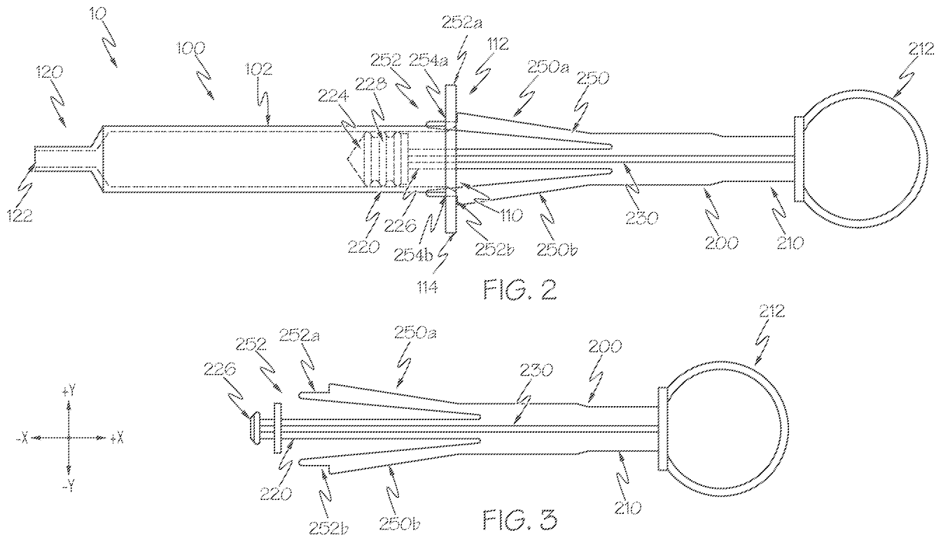

To perform a biopsy procedure, the aspiration accessory 10 may be moved from the initial position to the suction position, as is illustrated in FIG. 2. In these embodiments, to translate the aspiration accessory 10 from the initial position to the suction position, a user may insert a finger, such as a thumb, into the sliding mechanism 212 and pull the plunger 200 (e.g., in the +x direction illustrated by the coordinate axis of FIG. 2) away from the syringe barrel 100. As the plunger 200 is translated, the stopper 226 may similarly translate in the longitudinal direction (e.g., in the +x direction illustrated by the coordinate axis of FIG. 2), such that the stopper 226 no longer partially extends into the aperture 122.

Because a fluid-tight seal may exist between the stopper 226 and the body 102 of the syringe barrel 100, retraction of the plunger 200 (e.g., in the +x direction as illustrated by the coordinate axis of FIG. 2) may result in negative pressure accumulating within the body 102 of the syringe barrel 100. This negative pressure provide a suction force which allows for a fluid (e.g., tissues, cells, etc.) to be pulled into the body 102 of the syringe barrel 100 as the plunger 200 is retracted.

In these embodiments, the plunger 200 may be retracted (e.g., in the +x direction as illustrated by the coordinate axis of FIG. 2) until the aspiration accessory 10 is moved to the suction position. As depicted in FIG. 2, the suction position may be any position in which the stopper 226 is withdrawn from the aperture 122, such that negative pressure is generated within the body 102 of the syringe barrel 100. Furthermore, the plunger 200 may be retracted to a maximum suction position, in which negative pressure generated by the retraction of the plunger 200 causes a maximum volume of fluid to be drawn into the body 102 of the syringe barrel 100.

Referring now to FIGS. 2-4, the aspiration accessory is depicted in the suction position. In these embodiments, the plunger 200 may be retracted (e.g., moved in the −x direction as depicted in the coordinate axis of FIG. 2) until the first and second flange 250a, 250b are withdrawn from the body 102 of the syringe barrel 100. As the first and second flanges 250a, 250b are withdrawn from the body 102, the outward radial bias of the flanges 250a, 250b may cause the flanges 250a, 250b to extend away from the central portion 230 of the plunger 200 (e.g., in the +/−y direction as illustrated by the coordinate axis of FIG. 2).

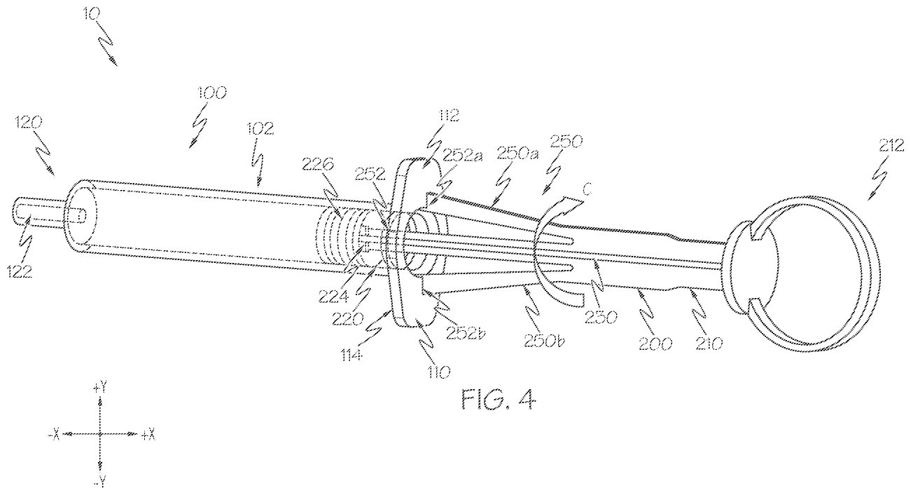

As the first and second flanges 250a, 250b are biased outwardly, the locking mechanisms 252a, 252b may contact the first end surface 112 of the first end 110 of the syringe barrel 100, as is most clearly depicted in FIG. 4. In these embodiments, the locking mechanisms 252a, 252b may each include a contact surface 254a, 254b, that face away from the sliding mechanism 212 (e.g., in the-x direction as depicted in the coordinate axis of FIG. 4). As the first and second flanges 250a, 250b, are biased outwardly, the contact surface 254a, 254b of each of the locking mechanisms 252a, 252b may engage the first end surface 112 of the first end 110 of the syringe barrel 100, such that longitudinal movement of the plunger 200 towards the second end 120 of the syringe barrel (e.g., in the-x direction as depicted by the coordinate axis of FIG. 4) is prevented.

Referring still to FIGS. 2-4, with the plunger 200 secured in the suction position, the aspiration accessory 10 may be rotated. For example, if a user has placed at least one finger, such as a thumb, through the sliding mechanism 212 to control the plunger 200 of the aspiration accessory, the user may utilize two additional fingers on the same hand to rotate the aspiration accessory 10 in a circumferential direction C. In these embodiments, the user may place the two additional fingers on the second end surface 114 of the first end 110 of the syringe barrel 100, such that the device is secured between the two additional fingers and the at least one finger engaging the sliding mechanism 212. Additionally, in some embodiments, once the plunger 200 is secured in the suction position, a user may remove at least one finger, such as a thumb, from the sliding mechanism 212 and grasp the barrel 102, at which point the user may rotate the barrel 102 in the circumferential direction to affect rotation of the aspiration accessory.

Once the biopsy procedure has been completed, the aspiration accessory 10 may be returned to the initial position to relieve the suction force acting within the syringe barrel 100. In these embodiments, a user may apply a manual compression force (e.g., using multiple fingers) to the first and second flanges 250a, 250b. As the flanges 250a, 250b are compressed, the locking mechanisms 252a, 252b may be unlocked, such that the locking mechanisms 252a, 252b move inwardly (e.g., in the +/−y direction as illustrated in the coordinate axis of FIG. 2). As the locking mechanisms 252a, 252b move inwardly, the contact surfaces 254a, 254b may disengage the first end surface 112 of the first end 110 of the syringe barrel 100, thereby allowing the first and second flanges 250a, 250b to move in a lateral direction (e.g., in the-x direction depicted in the coordinate axis of FIG. 2) and reenter the body 102 of the syringe barrel 100. Once the locking mechanisms 252a, 252b disengage the first end surface 112 of the first end 110 of the syringe barrel 100, the first and second flanges 250a, 250b, may be compressed and pushed (e.g., in the−x direction as depicted in the coordinate axis of FIG. 2) into the body 102 of the syringe barrel 100, thereby translating the plunger 200 through the body 102 towards the second end 120 of the syringe barrel 100. As the plunger 200 is forced through the syringe barrel 100, the motion of the plunger 200 may cause any fluid received by the syringe barrel 100 during the FNA biopsy procedure to be expelled from the syringe barrel 100 via aperture 122.

It should be understood that, in these embodiments, the volume of fluid drawn into the body 102 of the syringe barrel 100 may correspond to a radius of the syringe body 102 and a longitudinal distance which the plunger 200 is retracted between the initial position (FIG. 1) and the suction position (FIGS. 2-4). For example, the volume of fluid that may be contained within the body 102 may correspond to the radius of the body 102, such that by utilizing a syringe barrel 100 that has a body 102 with a larger radius, the volume of fluid that may be contained within the body 102 may be increased. Similarly, the longitudinal distance which the plunger 200 is retracted may further correspond to the volume of fluid drawn into the body, as a greater volume of fluid may be drawn into the body 102 of the syringe barrel 100 when the distance the plunger 200 is retracted increases.

For example, in the embodiments depicted herein, the syringe barrel 100 may have a volume of 30 mL. In these embodiments, the maximum volume of fluid that may be drawn into the body 102 of the syringe barrel 100 may be considered the volume of the syringe barrel 100 between the stopper 226 and the second end 120 of the syringe barrel 100. Because the stopper 226 is not fully withdrawn from the body 102 in the suction position (as depicted in FIG. 4), the stopper 226 may continue to occupy a portion of the volume of the syringe barrel 100 in the suction position. For example, the stopper 226 may be sized such that the stopper 226 occupies 5 mL of space within the body 102. Accordingly, in embodiments in which the syringe barrel has a volume of 30 mL, the maximum volume of fluid that may be drawn into the body 102 of the syringe barrel 100 may be 25 mL. Although the syringe barrel 100 is depicted as having a volume of 30 mL, it should be understood that the syringe barrel 100 may have any volume that may be utilized by a particular procedure without departing from the scope of the present disclosure. Similarly, it should be understood that, although the stopper 226 is depicted as occupying 5 mL of the body 102, the stopper 226 may be any size stopper 226 that can be positioned within the body 102 without departing from the scope of the present disclosure.

Operation of the aspiration accessory 10 will now be described in detail herein. For example, in the embodiments depicted herein, the plunger 200 is depicted as partially obstructing the aperture 122 of the second end 120 of the syringe barrel 100 in the initial position (FIG. 1). As a result, the plunger 200 must be retracted across the length of the body 102 of the syringe barrel 100 in order to achieve the suction position depicted in FIGS. 2-4. Because the plunger 200 traverses the body 102 of the syringe barrel 100 moving from the initial position to the suction position, the negative force generated by the plunger 200 within the body 102 may cause the body 102 to fill with fluid. For example, in embodiments in which the syringe barrel has a 30 mL volume, retraction of the plunger 200 from the initial position to the suction position may cause the body 102 of the syringe barrel 100 to be filled with 25 mL of fluid, as has been described herein above.

Accordingly, the aspiration accessory 10 may allow for a predetermined volume of fluid to be withdrawn from a target site by altering the position of the plunger 200 within the body 102 of the syringe barrel 100 in the initial position. For example, although the plunger 200 is depicted as partially obstructing the aperture 122 of the second end 120 of the syringe barrel 100 in the initial position (FIG. 1), the plunger 200 may be moved to a central location of the body 102 in the initial position. In these embodiments, the plunger 200 would only be retracted through a portion of the body 102 to move from the initial position to the suction position. By decreasing the distance in which the plunger 200 is retracted between the initial position and the suction position, the negative pressure generated by translation of the plunger 200 is similarly decreased. In turn, the volume of fluid drawn into the body 102 of the syringe barrel 100 is also decreased.

For example, in embodiments in which the syringe barrel 100 has a volume of 30 mL, the plunger 200 may be retracted such that the stopper is positioned at a center point of the body 102 of the syringe barrel 100 (e.g., equal distance from the first end 110 and the second end 120) in the initial position. In these embodiments, the plunger 200 may only traverse half of the length of the syringe barrel 100 as the plunger 200 is retracted from the initial position to the suction position. As a result, retraction of the plunger 200 from the initial position to the suction position may cause the body 102 of the syringe barrel 100 to be filled with approximately 12.5 mL of fluid, rather than the full 25 mL volume of the syringe barrel 100.

In view of the foregoing, it should be understood that the initial position of the plunger 200 may be adjusted to any position within the body 102 of the syringe barrel 100 in order to adjust the volume of fluid acquired by the aspiration accessory 10 during a FNA biopsy procedure. Furthermore, in these embodiments, the initial position of the plunger 200 may be adjusted prior to inserting the aspiration accessory 10 into a target site. By adjusting the initial position of the plunger 200 prior to inserting the aspiration accessory 10 into the target site, a user may ensure that the desired volume of fluid is acquired during the FNA biopsy process, as the plunger 200 locks in the suction position.

Although the initial position of the plunger 200 may be altered prior to inserting the aspiration accessory 10 into the target site in order to adjust the volume of fluid acquired by the aspiration accessory 10, in some embodiments, the aspiration accessory 10 may be inserted into the target site prior to adjusting the plunger 200. In these embodiments, a user may partially retract the plunger 200 in order to adjust the volume of fluid acquired by the aspiration accessory. However, in these embodiments, the locking mechanisms 252 of the plunger 200 may not lock the plunger 200 in the suction position when the plunger 200 is only partially retracted. Accordingly, it may be more difficult to achieve a desired fluid volume in embodiments in which the aspiration accessory 10 is inserted into the target site before the initial position of the plunger 200 is adjusted, as the plunger 200 may continue to move in the longitudinal direction throughout the body 102 of the syringe barrel 100 when the locking mechanisms 252 do not secure the plunger 200 in the suction position. For example, the negative pressure generated within the body 102 of the syringe barrel 100 as the plunger 200 is retracted may act to draw the plunger 200 towards the initial position (e.g., in the-x direction as depicted in the coordinate axes of FIGS. 2-4). However, this issue may be alleviated by engaging the locking mechanisms 252 of the plurality of flanges 250 with the first end surface 112 of the first end 110 of the syringe barrel 100.

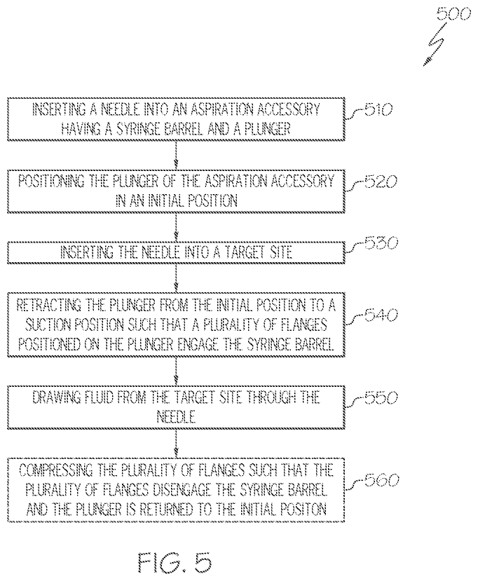

Turning now to FIG. 5, an illustrative flow diagram of a method 500 of performing an aspiration biopsy is depicted. Initially, as illustrated at block 510, the method may involve inserting a needle, such as a fine needle, into an aspiration accessory. In these embodiments, the aspiration accessory may include a syringe barrel having a first end surface and a plunger having a plurality of flanges that are biased radially outward. The plunger may be moved between an initial position and a suction position, and the plurality of flanges may be compressed within the syringe barrel in the initial position.

Once the needle has been coupled to the aspiration accessory, the method may move to block 520, which may involve positioning the plunger in the initial position such that the plunger is contained in the syringe barrel. In these embodiments, the distance the plunger retracts from the initial position and the suction position may correspond to a volume of fluid drawn into the syringe barrel. Accordingly, the initial position of the plunger may be adjusted to adjust the volume of fluid drawn into the syringe barrel during an aspiration biopsy procedure.

For example, increasing the distance the plunger retracts between the initial position and the suction position may increase the negative pressure generated by the plunger within the syringe barrel, such that the volume of fluid drawn from a target site increases. Conversely, decreasing the distance the plunger retracts between the initial position and the suction position may decrease the negative pressure generated by the plunger within the syringe barrel, thereby decreasing the volume of fluid drawn from the target site.

Referring still to FIG. 5, once the plunger has been moved to the initial position, the method may move to block 530, which may involve inserting the needle into the target site. With the needle positioned, the method may move to block 540, which may involve retracting the plunger from the initial position to the suction position. As the plunger is retracted from the initial position to the suction position, fluid may be drawn from the target site and into the syringe barrel via the needle. Similarly, as the plunger is retracted, a biasing force on the plurality of flanges may cause the plurality of flanges to flex radially outward once the plurality of flanges are retracted from the syringe barrel. As the plurality of flanges flex outwardly, the plurality of flanges may engage the first end surface of the syringe barrel to lock the plunger in the suction position.

As indicated at block 550, the method 500 may further involve drawing fluid from the target site and into the syringe barrel via the needle, as has been described herein. In these embodiments, it should be understood that block 540 of the method 500 of retracting the plunger and block 550 of the method 500 of drawing fluid from the target site may occur simultaneously or substantially simultaneously. That is, the retraction of the plunger from the initial position to the suction position may cause fluid to be drawn from the target site and into the syringe barrel.

In some embodiments, the method 500 may optionally include compressing the plurality of flanges to release the plunger form the suction position, such that the plunger translates laterally to the initial position as is shown at block 560. In these embodiments, applying a compressive force to the plurality of flanges may cause the plurality of flanges to disengage the first end surface of the syringe barrel such that the plunger may be returned to the initial position. Once the plunger is translated to the initial position, the accessory may be removed from the target site and the fluid within the syringe barrel may be dispensed.

As should be appreciated in view of the foregoing, an aspiration accessory for performing a biopsy procedure is disclosed. The aspiration accessory may include a syringe barrel having a first end including a first end surface and a second end. A plunger having a distal end and a proximal end may be slidably engaged within the syringe barrel between an initial position and a suction position, and a plurality of flanges may be positioned between the distal end of the syringe barrel and the proximal end of the syringe barrel. The plurality of flanges may be biased radially outward, such that the syringe barrel compresses the plurality of flanges in the initial position. When the plunger is moved to the suction position, a biasing force on the plurality of flanges may cause the plurality of flanges to flex radially outward, such that the plurality of flanges engage the first end surface of the syringe barrel and lock the aspiration accessory in the suction position. The aspiration accessory may be specifically advantageous for withdrawing a predetermined volume of fluid from a tissue site (e.g., by adjusting the initial position of the plunger) and may further be manually operated, such that the aspiration accessory may be easily rotated and positioned using a finger, such as a thumb or index finger, during a biopsy procedure.

Embodiments may be further described with references to the following numbered clauses:
1. An aspiration accessory, comprising: a syringe barrel having a first end and a second end, the first end including a first end surface; and a plunger slidably engaged within the syringe barrel between an initial position and a suction position, the plunger having a distal end and a proximal end, and a plurality of flanges positioned between the distal end and the proximal end, the plurality of flanges being biased radially outward, the syringe barrel compressing the plurality of flanges in the initial position; wherein, when the plunger is moved to the suction position, a biasing force on the plurality of flanges cause the plurality of flanges to flex radially outward and engage the first end surface of the syringe barrel, such that the plunger locks in the suction position.
2. The aspiration accessory of item 1, wherein the distal end of the plunger further comprises a finger ring.
3. The aspiration accessory of item 1 or 2, wherein the second end of the syringe barrel is coupled to a needle.
4. The aspiration accessory of any of items 1-3, wherein the plunger further comprises a stopper, the stopper being in a sealed sliding engagement with the syringe barrel.
5. The aspiration accessory of any of items 1-4, wherein a suction force is generated in the syringe barrel when the plunger is retracted from the initial position to the suction position.
6. The aspiration accessory of item 5, wherein the suction force generated in the syringe barrel is released when the plunger is returned from the suction position to the initial position.
7. The aspiration accessory of any of items 2-6, wherein the plurality of flanges are compressed to disengage the plurality of flanges from the first end surface of the syringe barrel, such that the plunger is unlocked and moved from the suction position to the initial position.
8. A biopsy device comprising: an aspiration accessory comprising: a syringe barrel having a first end and a second end, the first end including a first end surface; and a plunger being retractable between an initial position and a suction position, the plunger having a distal end and a proximal end, and a plurality of flanges positioned between the distal end and the proximal end, the plurality of flanges being biased radially outward, the syringe barrel compressing the plurality of flanges in the initial position and a biopsy needle coupled to the second end of the syringe barrel; wherein, when the plunger is moved to the suction position, a biasing force on the plurality of flanges cause the plurality of flanges to flex radially outward and engage the first end surface of the syringe barrel, such that the plunger locks in the suction position.
9. The biopsy device of item 8, wherein the distal end of the plunger further comprises a finger ring.
10. The biopsy device of item 8 or 9, wherein the biopsy needle is a fine aspiration needle.
11. The biopsy device of any of items 8-10, wherein the plunger further comprises a stopper, the stopper being in a sealed sliding engagement with the syringe barrel.
12. The biopsy device of any of items 8-11, wherein a suction force is generated in the syringe barrel when the plunger is retracted from the initial position to the suction position.
13. The biopsy device of any of items 8-12, wherein the plurality of flanges are compressed to disengage the plurality of flanges from the first end surface of the syringe barrel, such that the plunger is unlocked and moved from the suction position to the initial position.
14. The biopsy device of any of items 8-13, wherein the finger ring is slidable such that a user can retract the plunger from the initial position to the suction position with a single finger or thumb.
15. A method of performing an aspiration biopsy comprising: inserting a needle into an aspiration accessory, the aspiration accessory comprising a syringe barrel having a first end surface and a plunger having a plurality of flanges, the plurality of flanges being biased radially outward, the syringe barrel compressing the plurality of flanges in an initial position; positioning the plunger in the initial position, such that the plunger is contained within the syringe barrel; inserting the needle into a target site; retracting the plunger from the initial position to a suction position, such that a biasing force on the plurality of flanges cause the plurality of flanges to flex radially outward and engage the first end surface of the syringe barrel to lock the plunger in the suction position; and drawing fluid from the target site through the needle and into the syringe barrel.
16. The method of item 15, further comprising compressing the plurality of flanges to release the plunger from the suction position, such that the plunger is returned to the initial position.
17. The method of item 16, further comprising removing the needle from the target site and dispensing the fluid in the syringe barrel.
18. The method of any of items 15-17, wherein retracting the plunger from the initial position to the suction position generates a suction force in the syringe barrel.
19. The method of item 18, further comprising adjusting the plunger to achieve a desired level of suction force in the syringe barrel.
20. The method of any of items 15-19, wherein the plunger further includes a finger ring such that retracting the plunger from the initial position to the suction position is done manually with a single finger or thumb.

The terminology used herein is for the purpose of describing particular aspects only and is not intended to be limiting. As used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. An aspiration accessory, comprising:
a syringe barrel having a first end and a second end, the first end including a first end surface; and
a plunger slidably engaged within the syringe barrel between an initial position and a suction position, the plunger having a distal end and a proximal end, and a plurality of flanges positioned between the distal end and the proximal end, the plurality of flanges being biased radially outward, the syringe barrel compressing the plurality of flanges in the initial position;
wherein, when the plunger is moved to the suction position, a biasing force on the plurality of flanges cause the plurality of flanges to flex radially outward and engage the first end surface of the syringe barrel, such that the plunger locks in the suction position.

2. The aspiration accessory of claim 1, wherein the distal end of the plunger further comprises a finger ring.

3. The aspiration accessory of claim 1, wherein the second end of the syringe barrel is coupled to a needle.

4. The aspiration accessory of claim 1, wherein the plunger further comprises a stopper, the stopper being in a sealed sliding engagement with the syringe barrel.

5. The aspiration accessory of claim 1, wherein a suction force is generated in the syringe barrel when the plunger is retracted from the initial position to the suction position.

6. The aspiration accessory of claim 5, wherein the suction force generated in the syringe barrel is released when the plunger is returned from the suction position to the initial position.

7. The aspiration accessory of claim 2, wherein the plurality of flanges are compressed to disengage the plurality of flanges from the first end surface of the syringe barrel, such that the plunger is unlocked and moved from the suction position to the initial position.

8. A biopsy device comprising:
an aspiration accessory comprising:
a syringe barrel having a first end and a second end, the first end including a first end surface; and
a plunger being retractable between an initial position and a suction position, the plunger having a distal end and a proximal end, and a plurality of flanges positioned between the distal end and the proximal end, the plurality of flanges being biased radially outward, the syringe barrel compressing the plurality of flanges in the initial position; and
a biopsy needle coupled to the second end of the syringe barrel;
wherein, when the plunger is moved to the suction position, a biasing force on the plurality of flanges cause the plurality of flanges to flex radially outward and engage the first end surface of the syringe barrel, such that the plunger locks in the suction position.

9. The biopsy device of claim 8, wherein the distal end of the plunger further comprises a finger ring.

10. The biopsy device of claim 8, wherein the biopsy needle is a fine aspiration needle.

11. The biopsy device of claim 8, wherein the plunger further comprises a stopper, the stopper being in a sealed sliding engagement with the syringe barrel.

12. The biopsy device of claim 8, wherein a suction force is generated in the syringe barrel when the plunger is retracted from the initial position to the suction position.

13. The biopsy device of claim 8, wherein the plurality of flanges are compressed to disengage the plurality of flanges from the first end surface of the syringe barrel, such that the plunger is unlocked and moved from the suction position to the initial position.

14. The biopsy device of claim 9, wherein the finger ring is slidable such that a user can retract the plunger from the initial position to the suction position with a single finger or thumb.

15. A method of performing an aspiration biopsy comprising:
inserting a needle into an aspiration accessory, the aspiration accessory comprising a syringe barrel having a first end surface and a plunger having a plurality of flanges, the plurality of flanges being biased radially outward, the syringe barrel compressing the plurality of flanges in an initial position;
positioning the plunger in the initial position, such that the plunger is contained within the syringe barrel;
inserting the needle into a target site;
retracting the plunger from the initial position to a suction position, such that a biasing force on the plurality of flanges cause the plurality of flanges to flex radially outward and engage the first end surface of the syringe barrel to lock the plunger in the suction position; and
drawing fluid from the target site through the needle and into the syringe barrel.

16. The method of claim 15, further comprising compressing the plurality of flanges to release the plunger from the suction position, such that the plunger is returned to the initial position.

17. The method of claim 16, further comprising removing the needle from the target site and dispensing the fluid in the syringe barrel.

18. The method of claim 15, wherein retracting the plunger from the initial position to the suction position generates a suction force in the syringe barrel.

19. The method of claim 18, further comprising adjusting the plunger to achieve a desired level of suction force in the syringe barrel.

20. The method of claim 15, wherein the plunger further includes a finger ring such that retracting the plunger from the initial position to the suction position is done manually with a single finger or thumb.
